(19) United States
(12) Patent Application Publication
(10) Pub. No.: US 2009/0036708 A1
(43) Pub. Date: Feb. 5, 2009

(54) Novel Process for the Preparation of Nonracemic Long Chain alpha-Amino Acid Derivatives

(76) Inventors: Lanqi Jia, Ambler, PA (US); Rujian Ma, Shanghai (CN); Feng Zhang, Shanghai (CN); Yifeng Shi, Shanghai (CN); Jingchao Dong, Shanghai (CN); Ge Li, Shanghai (CN)

Correspondence Address: PAUL, HASTINGS, JANOFSKY & WALKER LLP, 875 15th Street, NW, Washington, DC, 20005, US

(21) Appl. No.: 11/577,089

(22) PCT Filed: Oct. 14, 2004
(86) PCT No.: PCT/CN04/01167

§ 371 Date: Oct. 10, 2007

(51) Int. Cl. C07C 229/30

(52) U.S. Cl. 562/553

(57) ABSTRACT

The present invention provides a process for the preparation of a nonracemic a-amino acid derivative from an optically active N-acyl lactam using an organometallic reagent to effect the opening of the ring followed by reduction of the ketone carbonyl to an alcohol or a methylene, or by a reductive amination to an amine, or by a ketalization reaction.

### FIELD OF THE INVENTION

This invention relates generally to the preparation of a nonracemic α-amino acid derivatives. This invention more specifically relates to preparing a nonracemic long chain chiral α-amino acid derivative from an optically active N-acyl lactam, using an organometallic reagent to open an N-acyl lactam followed by reduction of the ketone carbonyl to an alcohol or a methylene, or by reductive amination to an amine, or by a ketalization reaction.

### BACKGROUND OF THE INVENTION

α-Amino acid derivatives are useful as intermediates for the preparation of pharmaceutically active compounds. For example, the nonracemic compound, (L)-2-amino-non-8-enoic acid (Compound 1) is a key intermediate for the preparation of BILN-2061, a phase II clinical candidate for the treatment of Hepatitis C(HCV).

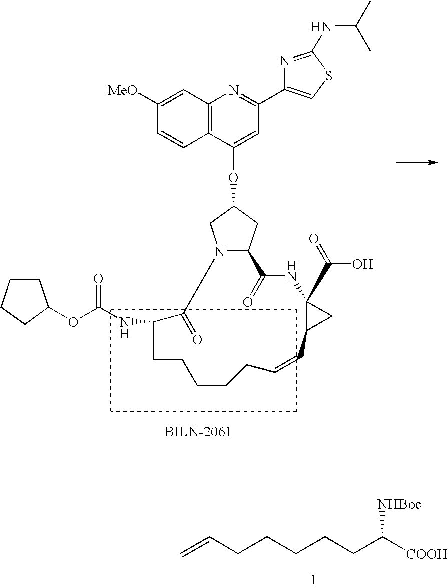

Other nonracemic α-amino acid derivatives have also found utility as biologically active species and as intermediates for the synthesis of other pharmaceutically active compounds. Nonracemic α-amino acid derivatives may be prepared by asymmetric synthetic routes to create the chiral center. See, e.g., WO 00/59929, WO 00/59929 and WO 03/064455.

One application of the current invention is for the synthesis of Compound 1 for use in the preparation of BILN-2061, which is of interest as a drug candidate. Two routes for preparing Compound 1 have been reported.

Even's method for making Compound 1, reported in WO 00/59929, synthesizes the chiral center with the assistance of a chiral auxiliary. That synthesis of Compound 1, (L)-2-amino-non-8-enoic acid, is illustrated in Route 1 below:

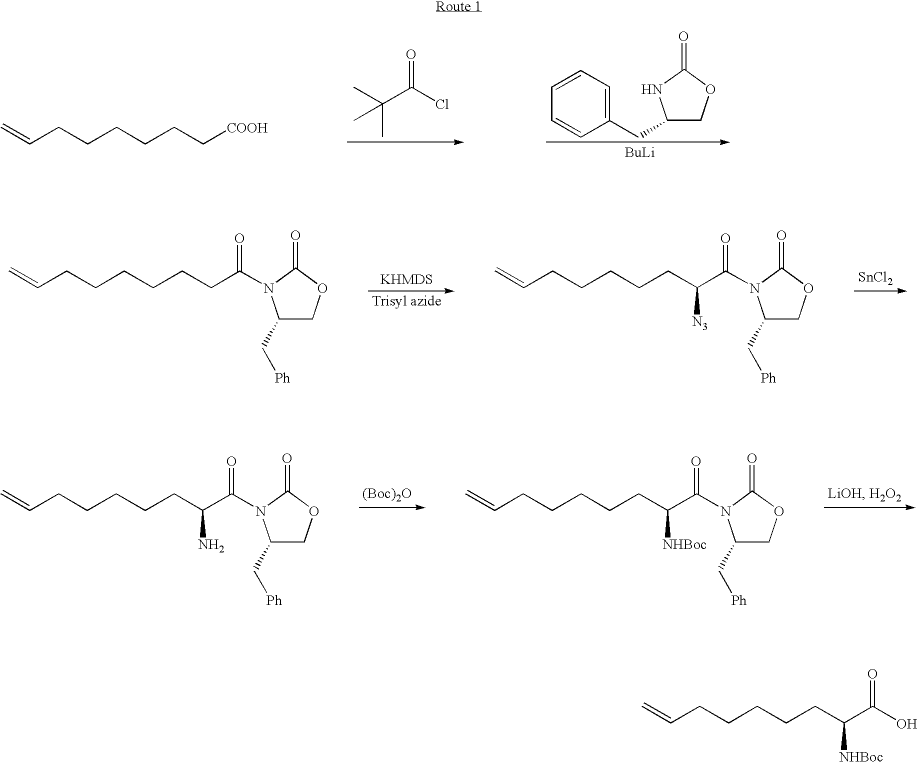

A second synthesis of Compound 1 is reported in WO 00/59929 and WO 03/064455, which utilize an asymmetric hydrogenation with the assistance of a chiral catalyst to introduce the chiral center of the amino acid. The synthesis of Compound 1, (L)-2-amino-non-8-enoic acid, by that method is illustrated in Route 2 below:

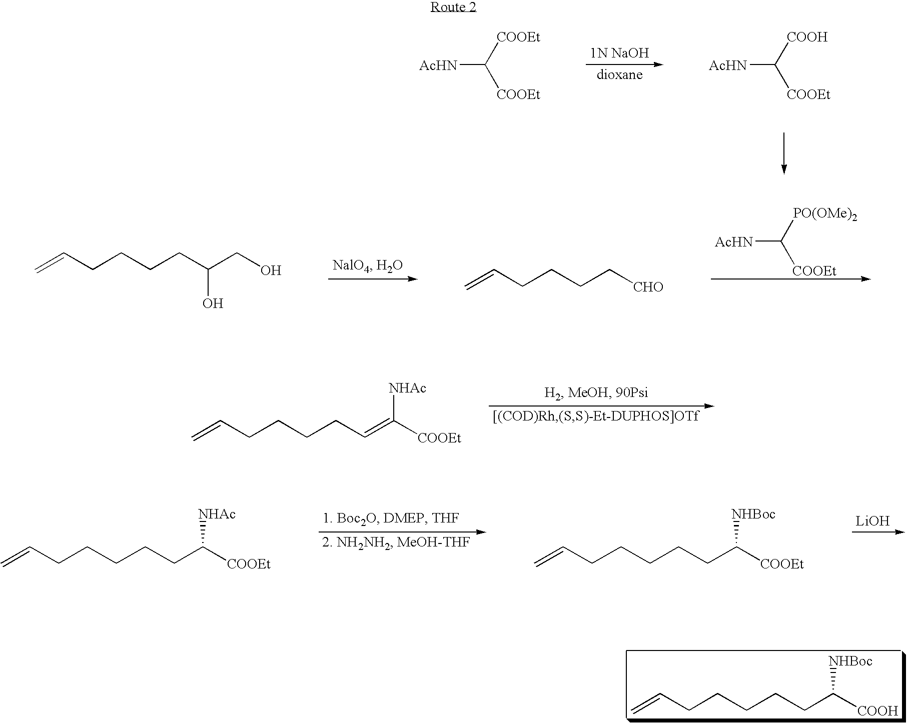

Both of the above methods are quite long and require reagents which are either expensive or difficult to make, use and recover; therefore are difficult and costly to practice on a commercial production scale. Accordingly, there is a need for a new synthetic strategy to prepare optically active α-amino acid derivatives. The present invention fulfills this need by providing a process for making Compound 1 that is more amenable to commercial scale production at reasonable manufacturing costs. The invention further provides access to analogs of Compound 1 useful for the preparation of biologically active analogs of BILN-2061.

### SUMMARY OF THE INVENTION

The present invention provides a process for the preparation of α-amino acid derivatives from N-acyl lactams. The process involves reaction of an N-acyl lactam with an organometallic reagent to effect a ring opening of the lactam, followed by a reduction of the ketone carbonyl that was formed in the ring opening reaction. The process is surprisingly efficient, in that the reduction of the ketone carbonyl to a methylene can be accomplished in good yield via a two step process involving an intermediate sulfonyl hydrazone that requires no isolation of the intermediate. The inventors also have found that when starting with an optically active N-acyl lactam, the products are obtained in unexpectedly high optical purity: the optical purity of the product approximately matches that of the starting lactam.

In one aspect, this invention provides a process to prepare nonracemic α-amino acid with the structure:

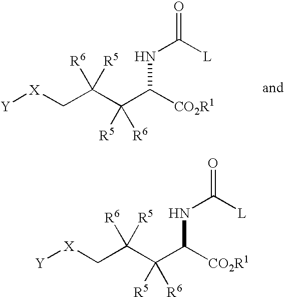

wherein:
R¹ is H, optionally substituted C₁-C₆ alkyl or optionally substituted C₇-C₁₂ aralkyl;
R⁵ and R⁶ are independently selected from the group consisting of H, optionally substituted C₁-C₆ alkyl, optionally substituted C₁-C₆ alkoxy, optionally substituted C₇-C₁₂ aralkyl and heteroaralkyl, and optionally substituted phenyl;
R⁷ are optionally substituted C₁-C₆ alkyl, optionally substituted C₁-C₆ alkoxy, optionally substituted C₇-C₁₂ aralkyl, or optionally substituted phenyl;
R⁹ is selected from the group consisting of optionally substituted C₁-C₆ alkyl, optionally substituted C₇-C₁₂ aralkyl, and —SiR⁵R⁶R⁷;
L is selected from the group consisting of tert-butoxy, optionally substituted C₁-C₆ alkoxy, and optionally substituted C₇-C₁₂ aralkyloxy;
X—Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4, n=0 to 2, and
Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted C₁-C₆ alkyl optionally substituted aryl, optionally substituted heteroaryl, optionally substituted heterocycle, and —C≡C—R⁹.

In another aspect, this invention provides a novel practical general process to prepare each optical isomer of long chain terminal-olefin α-amino acids, which have the structure:

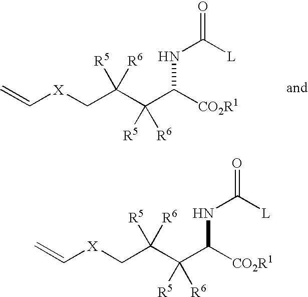

wherein:
R¹ is H, optionally substituted C₁-C₆ alkyl or optionally substituted C₇-C₁₂ aralkyl;
R⁵ and R⁶ are independently selected from the group consisting of H, optionally substituted C₁-C₆ alkyl, optionally substituted C₁-C₆ alkoxy, optionally substituted C₇-C₁₂ aralkyl and heteroaralkyl, and optionally substituted phenyl;
L is selected from the group consisting of tert-butoxy, optionally substituted C₁-C₆ alkoxy, and optionally substituted C₇-C₁₂ aralkyloxy;
X is selected from the group consisting of —(CH₂)ₘ—, —CH₂O—, —CH₂S(O)ₙ—, —CH₂CH₂CH₂O—, —CH₂CH₂CH₂S(O)ₙ—, —CH₂—O—(CH₂)ₘ—, and —CH₂S(O)ₙ(CH₂)ₘ—, wherein m=0 to 4, n=0 to 2.

In another aspect of the invention, the ring-opening reaction is followed by conversion of the ketone carbonyl into another group such as a ketal, thioketal, animal, hemiaminal, or dithioketal, or an oxime or alkoxyimines by methods known in the art. The ketone carbonyl can also be converted to an optionally substituted amine by reductive amination.

One aspect of the invention is a process for the preparation of a nonracemic amino acid derivative from an optically active N-acyl lactam of Formula I,

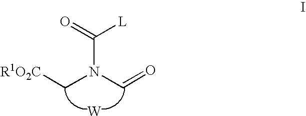

comprising the steps of:
(i) adding an organometallic reagent M-X-Y to a compound of Formula I dissolved in an ethereal solvent to produce a compound of Formula II,

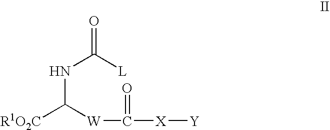

wherein:
   M is MgCl, MgBr, MgI, or Li;
   R¹ is optionally substituted C₁-C₆ alkyl or optionally substituted C₇-C₁₂ aralkyl;
   R⁵ and R⁶ is independently selected from the group consisting of H, optionally substituted C₁-C₆ alkyl, optionally substituted C₁-C₆ alkoxy, optionally substituted C₇-C₁₂ aralkyl, and optionally substituted phenyl;
   R⁷ is optionally substituted C₁-C₆ alkyl, optionally substituted C₁-C₆ alkoxy, optionally substituted C₇-C₁₂ aralkyl, or optionally substituted phenyl;
   R⁹ is selected from the group consisting of optionally substituted C₁-C₆ alkyl, optionally substituted C₇-C₁₂ aralkyl, and —SiR⁵R⁶R⁷;
   L is selected from the group consisting of tert-butyl, optionally substituted C₁-C₆ alkoxy, and optionally substituted C₇-C₁₂ aralkyloxy;
   W is —(CR⁵R⁶)ₙ—, wherein n=2-4;
   X-Y is chosen from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4, n=0 to 2;
   Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹; and
(ii) reducing the ketone carbonyl of the compound of Formula II to produce a compound of Formula III,

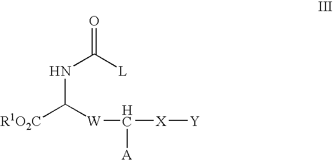

wherein A is H or OH.

In one embodiment, the ketone carbonyl reduction is effected by using a sulfonyl hydrazine reagent to form a sulfonyl hydrazide intermediate, which is then removed by reduction with a reducing reagent to produce a compound of Formula III wherein A is H. In one embodiment, the reducing reagent is a borohydride salt, and in an especially preferred embodiment the reducing reagent is a salt of triacetoxyborohydride. Another embodiment utilizes an arylsulfonyl hydrazine reagent to form the sulfonyl hydrazide intermediate, and exemplary arylsulfonyl hydrazine reagents include, but are not limited to, phenylsulfonyl hydrazine and substituted phenylsulfonyl hydrazines such as toluenesulfonyl hydrazine.

In another aspect of this invention, the ketone carbonyl formed by addition of an organometallic reagent to the N-acyl lactam is then reduced to an alcohol of Formula III, where A is OH. In one embodiment, such reduction is accomplished by treatment with a borohydride reagent such as sodium borohydride. In another embodiment, a chiral borohydride reagent is used in order to control the stereochemistry of the newly formed chiral alcohol center. In one embodiment of this aspect, W is —CH₂CH₂, X is —(CH₂)ₘ— and Y is unsubstituted vinyl or —C≡C—R⁹.

In another aspect of the invention, the ketone carbonyl is reduced to an alcohol group, which is then optionally converted into different substituents such as an ester (—OC(O)R²), carbamate (—OC(O)NR³R⁴), carbonate (—OC(O)OR²), halogen, alkyl or arylsulfonate (—OS(O)₂R²), silyl ether (—OSiR⁵R⁶R⁷), or amine (—NR³R⁴). In one embodiment, chiral reducing reagents are used to give chiral alcohols by stereoselective reductions.

In this aspect, the invention provides a process for producing compounds of Formula III wherein A is a member selected from the group consisting of halogen, —OR², —SR², —NR³R⁴, —N₃, —OSO₂R⁵ and —CN, where R² is selected from the group consisting of —C(O)R⁵, —C(O)OR⁵, —C(O)NR³R⁴, and —SiR⁵R⁶R⁷, wherein R³ and R⁴ are independently selected from the group consisting of H, optionally substituted C₁-C₆ alkyl, optionally substituted C₁-C₆ alkoxy, optionally substituted C₇-C₁₂ aralkyl, and optionally substituted phenyl, or R³ and R⁴ taken together with the N to which they are attached form a five to seven membered ring optionally including one additional heteroatom selected from the group consisting of O and S. Such compounds are produced from the compounds of Formula III where A is —OH by various manipulations of the hydroxyl group.

In one embodiment of this aspect, X is an alkylene group such as —(CH₂)ₘ— where m is an integer from 0 to 4. In other embodiments, Y is an unsubstituted vinyl group or an acetylene of formula —C≡C—R⁹, where R⁹ is defined as above, and other specific embodiments often include an unsubstituted vinyl group for Y. In another embodiment, W is an ethylene group with 0 to 2 substituents, having the formula —CHR⁵CHR⁶—. In yet another embodiment, R⁵ and R⁶ are independently H or C₁-C₆ alkyl groups. In still yet another preferred embodiment, W is an ethylene group, —CH₂CH₂—.

In one embodiment, L is benzyloxy group and in another embodiment, L is a C₁-C₆ alkoxy group. Often, L is a tert-butoxy group. In one embodiment, X is —(CH₂)ₘ—, Y is unsubstituted vinyl or —C≡C—R⁹, where R⁹ is defined as above, and W is —CHR⁵CHR⁶— while L is benzyloxy or a C₁-C₆ alkoxy group. In another embodiment, W is —CH₂CH₂— and L is tert-butoxy, and in one of the specific embodiments, X is —(CH₂)₂— and Y is an unsubstituted vinyl group.

The process of the present invention can be used to produce racemic products where the N-acyl lactam starting material is racemic. However, where the N-acyl lactam is nonracemic, the invention provides products with optical activity. The optical activity of the product generally reflects that of the starting material: the process does not substantially affect the optical purity of the alpha-amino acid chiral center. Thus in one embodiment, the process of the present invention utilizes a nonracemic N-acyl lactam of Formula I to produce-nonracemic products. In another embodiment, the product of Formula III is produced in at least about 85% enantiomeric excess at the chiral alpha-amino acid center.

In another aspect, the invention provides a process for the preparation of a nonracemic amino acid derivative of Formula IV:

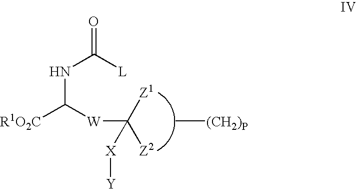

from an optically active N-acyl lactam of Formula I, comprising the steps of:
(i) adding an organometallic reagent M-X-Y to a compound of Formula I

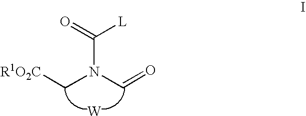

dissolved in an ethereal solvent to produce a compound of Formula II,

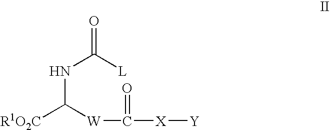

wherein:
   M is MgCl, MgBr, MgI, or Li;
   R¹ is optionally substituted C₁-C₆ alkyl or optionally substituted C₇-C₁₂ aralkyl;
   R⁵ and R⁶ is each independently selected from the group consisting of H, optionally substituted C₁-C₆ alkyl, optionally substituted C₁-C₆alkoxy, optionally substituted C₇-C₁₂ aralkyl, and optionally substituted phenyl;
   R⁷ is optionally substituted C₁-C₆ alkyl, optionally substituted C₁-C₆ alkoxy, optionally substituted C₇-C₁₂ aralkyl, or optionally substituted phenyl;
   R⁹ is selected from the group consisting of optionally substituted C₁-C₆ alkyl, optionally substituted C₇-C₁₂ aralkyl, and —SiR⁵R⁶R⁷;
   L is selected from the group consisting of tert-butyl, optionally substituted C₁-C₆ alkoxy, and optionally substituted C₇-C₁₂ aralkyloxy;
   W is —(CR⁵R⁶)ₙ— where n=2-4;
   X-Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and CH₂S(O)ₙ(CH₂)ₘ—Y, wherein m=0 to 4 and n=0 to 2;
   Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹; and
(ii) converting the ketone carbonyl of the compound of Formula II to a heterocyclic derivative of Formula IV, wherein Z¹ and Z² are independently selected from the group consisting of O, S, and NR¹¹, wherein R¹¹ is selected from the group consisting of H, C₁-C₆ alkyl, C₁-C₆ alkoxycarbonyl, and C₂-C₇ acyl, and p=2-4.

In one embodiment of this aspect of the invention, W is —CHR⁵CHR⁶— and X is —(CH₂)ₘ— in the product of Formula IV. In another embodiment, L is C₁-C₆ alkoxy, and in another embodiment, Y is unsubstituted vinyl or —C≡C—R⁹. In one specific embodiment,

W is —CH₂CH₂—. And in another specific embodiment, the compound of Formula IV is produced in at least about 85% e.e. at the chiral α-amino acid center.

While the invention provides a process for making nonracemic amino acid derivatives, in one aspect, in a broader aspect it provides a process for the preparation of an amino acid derivative from an N-acyl lactam of Formula I,

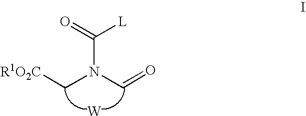

comprising the steps of:
(i) adding an organometallic reagent M-X-Y to a compound of Formula I dissolved in an ethereal solvent to produce a compound of Formula II,

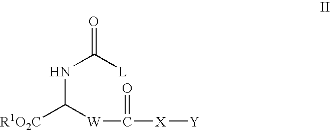

wherein:
   M is MgCl, MgBr, MgI, or Li;
   R¹ is optionally substituted C₁-C₆ alkyl or optionally substituted C₇-C₁₂ aralkyl;
   R⁵ and R⁶ is each independently selected from the group consisting of H, optionally substituted C₁-C₆alkyl, optionally substituted C₁-C₆ alkoxy, optionally substituted C₇-C₁₂ aralkyl, and optionally substituted phenyl;
   R₇ is optionally substituted C₁-C₆ alkyl, optionally substituted C₁-C₆ alkoxy, optionally substituted C₇-C₁₂ aralkyl, or optionally substituted phenyl, and R⁵ and R⁶ are as defined above;
   R⁹ is selected from the group consisting of optionally substituted C₁-C₆ alkyl, optionally substituted C₇-C₁₂ aralkyl, and —SiR⁵R⁶R⁷;
   L is selected from the group consisting of tert-butyl, optionally substituted C₁-C₆ alkoxy, and optionally substituted C₇-C₁₂ aralkyloxy;
   W is —(CR⁵R⁶)ₙ—, wherein n=2-4;
   X-Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4, n=0 to 2;
   Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹; and
(ii) reducing the ketone carbonyl of the compound of Formula II to produce a compound of Formula III,

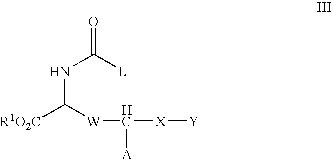

wherein A is H or OH.

In this aspect of the invention, the product may be either racemic or nonracemic at the chiral alpha-amino acid center; its enantiomeric excess approximately matches that of the N-acyl lactam starting material used for the first step of the process. Compounds of Formula III where A is not H have a second chiral center, hence they exist as diastereomers. The present invention provides a method to produce each diastereomer of such compounds.

### DETAILED DESCRIPTION OF THE INVENTION

### Definition

Unless otherwise stated, the following terms used in the specification and claims have the meanings given below:

“Alkyl” refers to a linear saturated monovalent hydrocarbon radical or a branched saturated monovalent hydrocarbon radical or a cyclic saturated monovalent hydrocarbon radical, having the number of carbon atoms indicated in the prefix. For example, C₁-C₆ alkyl is meant to include methyl, ethyl, n-propyl, 2-propyl, tert-butyl, pentyl, cyclopentyl, cyclohexyl and the like. For each of the definitions herein (e.g. alkyl, alkenyl, alkoxy, aralkyloxy), when a prefix is not included to indicate the number of main chain carbon atoms in an alkyl portion, the radical or portion thereof will have twelve or fewer main chain carbon atoms. A divalent alkyl radical refers to a linear saturated divalent hydrocarbon radical or a branched saturated divalent hydrocarbon radical having the number of carbon atoms indicated in the prefix. For example, a divalent C₁-C₆ alkyl is meant to include methylene, ethylene, propylene, 2-methylpropylene, pentylene, and the like.

“Alkenyl” means a linear monovalent hydrocarbon radical or a branched monovalent hydrocarbon radical having the number of carbon atoms indicated in the prefix and containing at least one double bond. For example, C₁-C₆ alkenyl is meant to include, ethenyl, propenyl, and the like.

“Alkynyl” means a linear monovalent hydrocarbon radical or a branched monovalent hydrocarbon radical containing at least one triple bond and having the number of carbon atoms indicated in the prefix. For example, C₁-C₆ alkynyl is meant to include ethynyl, propynyl, and the like.

“Alkoxy”, “aryloxy”, “aralkyloxy”, or “heteroaralkyloxy” means a radical —OR where R is an alkyl, aryl, aralkyl, or heteroaralkyl respectively, as defined herein, e.g., methoxy, phenoxy, benzyloxy, pyridin-2-ylmethyloxy, and the like.

“Aryl” means a monocyclic or bicyclic aromatic, hydrocarbon radical of 6 to 10 ring atoms which is optionally substituted independently with substituents selected from alkyl, alkenyl, alkynyl, halo, nitro, cyano, hydroxy, alkoxy, amino, mono-alkylamino, di-alkylamino and heteroalkyl. More specifically the term aryl includes, but is not limited to, phenyl, biphenyl, 1-naphthyl, and 2-naphthyl, and the derivatives thereof.

“Aralkyl” refers to a radical wherein an aryl group is attached to an alkyl group, the combination being attached to the remainder of the molecule through the alkyl portion. Examples of aralkyl groups are benzyl, phenylethyl, naphthylmethyl, and the like.

“Heteroalkyl” means an alkyl radical as defined herein with one, two or three substituents independently selected from cyano, alkoxy, amino, mono- or di-alkylamino, thioalkoxy, and the like, with the understanding that the point of attachment of the heteroalkyl radical to the remainder of the molecule is through a carbon atom of the heteroalkyl radical.

“Heterocycle” or “heterocyclic” refers to monocyclic or bicyclic ring structure wherein at least one heteroatom selected from O, N and S is contained in a non-aromatic ring consisting of up to 8 ring atoms, provided that the ring contains not more than 3 such heteroatoms, and that no two heteroatoms in a ring are bonded directly to each other.

“Heteroaryl” means a monocyclic or bicyclic radical of 5 to 15 ring atoms having at least one aromatic ring containing one, two, or three ring heteroatoms selected from N, O, and S, the remaining ring atoms being C, with the understanding that the attachment point of the heteroaryl radical will be on an aromatic ring. The heteroaryl ring is optionally substituted independently with one to four substituents, preferably one or two substituents, selected from alkyl, halo, nitro, cyano, hydroxy, alkoxy, amino, acylamino, mono-alkylamino, di-alkylamino, heteroalkyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, and the derivatives thereof.

“Heteroaralkyl” refers to a radical wherein an heteroaryl group (as defined above) is attached to an alkyl group, the combination being attached to the remainder of the molecule through the alkyl portion. Examples of heteroaralkyl groups are 2-pyridylmethyl, 3-thienylethyl, and the like.

The terms “alkyl, alkenyl, alkynyl, alkoxy, aryloxy, aralkyloxy, heteroaralkyloxy, aralkyl, aryl, heteroalkyl, heterocyclic and heteroaryl” include optionally substituted alkyl, alkenyl, alkynyl, alkoxy, aryloxy, aralkyloxy, heteroaralkyloxy, aralkyl, aryl, heteroalkyl, heterocyclic and heteroaryl groups.

The terms “optional” or “optionally” mean that the subsequently described event or circumstance may but need not occur, and that the description includes instances where the event or circumstance occurs and instances in which it does not. For example, “aryl optionally mono- or di-substituted with an alkyl” means that the alkyl may but need not be present, or either one alkyl or two may be present, and the description includes situations where the aryl is substituted with one or two alkyls and situations where the aryl is not substituted with an alkyl.

“Optionally substituted” groups may be substituted or unsubstituted. The substituents on any other “optionally substituted” groups may include, without limitation, one or more substituents independently selected from the group of alkyl, alkenyl, alkynyl, halo, nitro, cyano, hydroxy, alkoxy, amino, mono-alkylamino, di-alkylamino and heteroalkyl. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

Where a chiral center is present, the invention contemplates that each enantiomer or diastereomer can be prepared and isolated as desired. A property of the present invention is that starting with high optical purity at the chiral amino acid carbon center allows preparation of the desired products in almost equally high optical purity. Thus the process is equally applicable to R and S enantiomers of the amino acid-derived N-acyl lactam starting materials, and both enantiomers are included within the scope of the invention even though one enantiomer is often used for illustrations and examples.

Where the process produces additional chiral centers, it is understood that either or both enantiomers of the new chiral center may be obtained either by diastereoselective reaction conditions to form one of the enantiomers of the new center, or by preparation of a mixture of diastereomers at the new center followed by separation of the diastereomers by methods commonly used in the art. Thus the invention includes each diastereomer as well as mixtures of diastereomers where a new chiral center is introduced by the claimed process.

The present invention provides efficient methods for producing useful amino acid derivatives in high optical purity, so the optical purity of starting materials and products is sometimes described herein in terms of e.e. Enantiomeric excess (e.e.) is a conventional method for expressing the optical purity of a mixture containing two enantiomers of a molecule in unequal amounts. The e.e. of such a mixture where the R enantiomer dominates, for example, is calculated as: e.e. =(% R−% S)/(% R+% S), where % R represents the percentage of the R enantiomer present in the mixture, and % S represents the percentage of the S enantiomer present. Where multiple chiral centers are present, it is sometimes convenient still to describe the optical purity of the α-amino acid chiral center in terms of its “e.e.”, even though diastereomers are present. In that case, % R there would include all diastereomers present that have the R configuration at the α-amino acid center, and % S would include all diastereomers present where the α-amino acid center is of the S configuration.

The present invention further contemplates a convenient method for preparing racemic mixture of α-amino acid derivatives. The inventors have found that the optical property of the result of product matches the starting material: Starting with a racemic mixture of N-acyl lactam, the resultant α-amino acid is also a racemic mixture.

### Methodology

Methods for preparing the N-acyl lactams used in the present invention are well-known in the art. Such a N-acyl lactams is represented by Formula I.

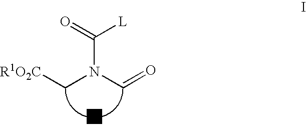

Many of the N-acyl lactams needed for the claimed process are available in high optical purity, e.g. with an enantiomeric excess (e.e.) of at least about 85%, and often with an e.e. of 90% or 95% or higher, and the optical activity of the product depends on the optical activity of the starting material. One of the advantages of the present invention is that it allows the optical activity of the chiral α-amino acid center to be retained without significant racemization as the material is converted into the desired product. Thus, using the claimed process, the desired α-amino acid derivatives are generally obtained with an optical purity that is about the same as that of the starting N-acyl lactam. Where the N-acyl lactam has an e.e. of 95% or higher, the product will generally have an e.e. of at least about 85%, often at least 90%, and preferably at least 95%. Where other chiral centers are introduced by the process, as for example, in compounds of Formula III

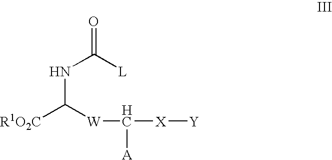

where A is not H, a mixture of diastereomers will be produced; nevertheless, the α-amino acid center will retain its original configuration without being substantially racemized, so the product will generally have an e.e. for the α-amino acid center of at least about 85% or higher.

Many of the organometallic reagents M-X-Y required for the present invention are well known in the art. Generally such reagents are prepared using standard conditions, for example, where the alkyl halide is available Grignard reagents are prepared using activated Mg metal in anhydrous ethereal solvents. Others are prepared from alkyl bromides or alkyl iodides by metal-halogen exchange reactions, using n-butyllithium, sec-butyllithium or tert-butyllithium. Some, such as where X—Y═CH₂O—Y, for example, are conveniently prepared by transmetallation of the corresponding organostannanes using butyllithium. Still others are prepared by deprotonation of appropriate precursors. For example, L₁-CH₂S-phenyl can be prepared directly by deprotonation of thioanisole (CH₃S-Phenyl) under conditions well known in the literature. Similarly, many aryllithium species may be prepared by direct deprotonation of corresponding arene rings, where deprotonation occurs adjacent to an ortho-metallation directing group on the arene starting material. Examples of this include metallation of 4-chloropyridine at the 3-position, and of O-methoxymethyl phenol ethers at a ring carbon adjacent to the methoxymethyl ether substituent. Other methods and the means to select which method to use for preparation of the specific organometallic reagent needed for making a particular target will be apparent to one of ordinary skill in the art. Table 1 below tabulates prior art methods of preparation the organometallic reagents.

TABLE 1

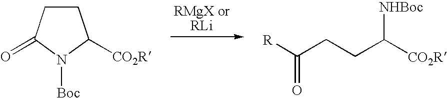

| Entry | RMgX or RLi | Refs |
|---|---|---|
| 1 | MeMgI | CL, 1987, 2091-2094 |
| 2 | 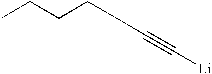 | CL, 1987, 2091-2094 |
| 3 | 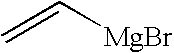 | CL, 1987, 2091-2094 |
| 4 | 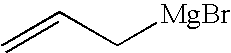 | CL, 1987, 2091-2094 |
| 5 | EtMgBr | TL, 1993, 34, 6317-6320 |
| 6 | PhMgBr and other ArMgBr. | TL, 1993, 34, 6317-6320 |
| 7 | 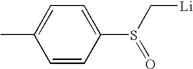 | TL, 1993, 34, 4989-4992 |
| 8 | 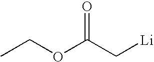 | TL, 1992, 33, 5589-5590 |
| 9 | 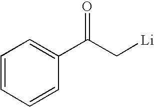 | TL, 1992, 33, 5589-5590 |
| 10 | 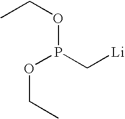 | TL, 1992, 33, 5589-5590 |
| 11 | 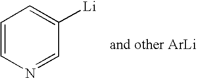 | JOC, 1999,64 4069-4078 |
| 12 | 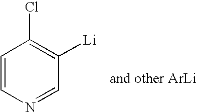 | JOC, 65, 961-870 |
| 13 | 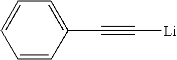 | Chem. Commun., 1997, 1757-1758 |
| 14 | 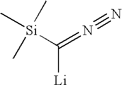 | TL, 1998, 39, 3242-3246; Synthesis, 2001,247-250 |
| 15 | 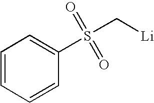 | Eur. J. Org. Chem., 2003, 4187-4198; TL,2003, 44, 1141-1144 |
| 16 | 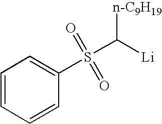 | Eur. J. Org. Chem., 2003, 4187-4198 |

Reactions of the N-acyl lactams with these organometallic reagents effect the ring opening and result in the formation of a ketone which is typified by Formula II.

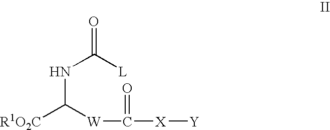

The reactions are typically conducted at a temperature well below 0° C. to ensure that the reaction occurs selectively at the desired amide linkage rather than at the ester center, and to prevent formation of byproducts derived from addition of the organometallic reagent to the ketone produced by the intended reaction. At low temperatures, such undesired reactions are usually minimized. Furthermore, the reaction must be conducted at a temperature where the organometallic reagent is itself stable enough to be synthetically useful. Typically, these reactions are conducted at −40° C. to −50° C. for from one hour to twelve hours, though temperatures of −78° C. to 0° C. may be used. Appropriate solvents for the reaction include THF, ether, DME, dioxane, or similar ethereal solvents or mixtures of these with anhydrous hydrocarbon solvents such as hexane.

Where the desired nonracemic α-amino acid is of Formula III where A=H, the ketones obtained by the above reaction are readily reduced to compounds of Formula III in a two step reaction that can conveniently be conducted in one pot. The ketone is first allowed to react with a sulfonyl hydrazine such as tosyl hydrazine to form a tosyl (or similar) hydrazone, which may occur in either a protic solvent such as acetic acid or an alcohol (e.g., methanol, ethanol, isopropanol, tert-butanol and the like), or an aprotic solvent such as hexane, or a mixture of protic and aprotic solvents such as THF plus water. The reaction typically occurs at room temperature or a higher temperature up to the reflux temperature of the solvent. Table 2 includes other prior art schemes to achieve this step of the invention.

TABLE 2

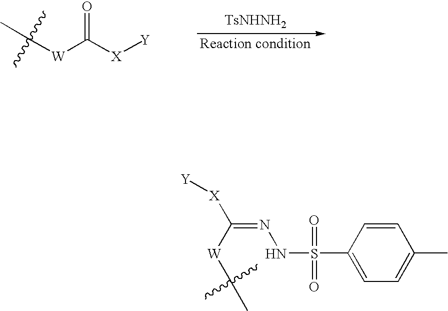

| Entry | Reaction condition | |
|---|---|---|
| 1 | Alcohol, heat | |
| 2 | Alcohol, heat, p-TSA | |
| 3 | Alcohol, aq. HCl | |
| 4 | Acetic acid | J. Am. Chem. Soc., 1954, 76, 4013-4024 |
| | | Tetrahedron, 1997, 53, 9989-9996 |
| 5 | Aq. HCl/THF | Tetrahedron, 1994, 50, 4399, 4428 |
| 6 | Hexane, 50° C. | Synthesis, 1996, 249-252. |

While these hydrazones can be isolated, more conveniently they are then reduced without isolation by the addition of a borohydride reagent such as sodium borohydride, sodium cyanoborohydride, or sodium triacetoxyborohydride, or by adding a borane such as catecholborane, using conditions known in the art for the particular reagent. Typically this reaction requires the use of 2 to 6 equivalents of the reducing agent and occurs over a period of from a few hours to about 5 days at the reflux temperature of the solvent employed. The reduced product is then isolated by conventional separation and purification methods. Table 3 includes several prior art scheme to effect the reduction.

TABLE 3

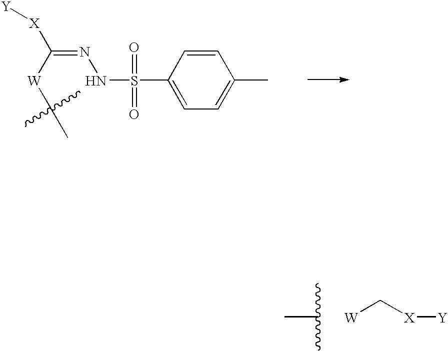

| Entry | Reaction condition | Ref: |
|---|---|---|
| 1 | NaBH₄/EtOH, heat | Syn. Lett, 1999, 1304-1306. |
| 2 | NaBH(OAc)₃, HOAc | Russ. J. Org. Chem., 1999, 35, |
| | | 1436-1438; |
| 3 | NaBH₃CN, p-TSA | Eur. J. Org. Chem., 1999; 2201-2210. |
| 4 | Catecholborane/ | Angew. Chem., 1999; 3554 3556; |
| | CH₃Cl/Toluene | Tetrahedron, 1999, 55; 7191-7208. |

Scheme 1 illustrates the general synthetic steps:

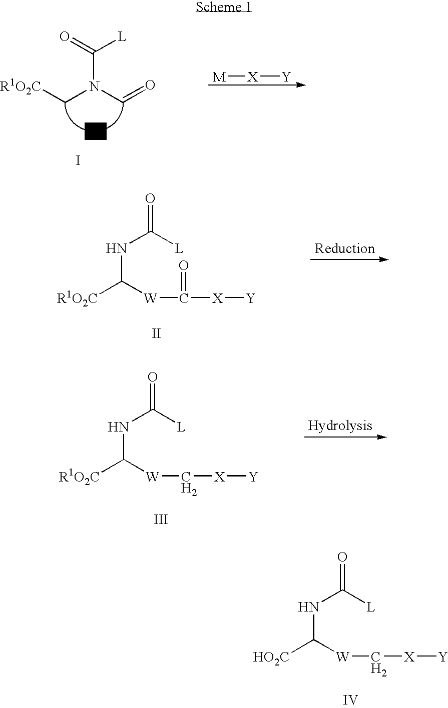

wherein
M is MgCl, MgBr, MgI, or Li;
R¹ is optionally substituted C₁-C₆ alkyl or optionally substituted C₇-C₁₂ aralkyl;
R⁵ and R⁶ is each independently selected from the group consisting of H, optionally substituted C₁-C₆ alkyl, optionally substituted C₁-C₆ alkoxy, optionally substituted C₇-C₁₂ aralkyl, and optionally substituted phenyl;
R⁷ is optionally substituted C₁-C₆ alkyl optionally substituted C₁-C₆ alkoxy, optionally substituted C₇-C₁₂ aralkyl, or optionally substituted phenyl;
R⁹ is selected from the group consisting of optionally substituted C₁-C₆ alkyl, optionally substituted C₇-C₁₂ aralkyl, and —SiR⁵R⁶R⁷;
L is selected from the group consisting of tert-butyl, optionally substituted C₁-C₆ alkoxy, and optionally substituted C₇-C₁₂ aralkyloxy;
W is —(CR⁵R⁶)ₙ—, wherein n=2-4;
X-Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and CH₂S(O)ₙ(CH₂)ₘ—Y, wherein m=0 to 4, n=0 to 2, and
Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹.

In one embodiment of the invention, nonracemic long chain α-amino acids bearing terminal olefin is prepared according to Scheme 2:

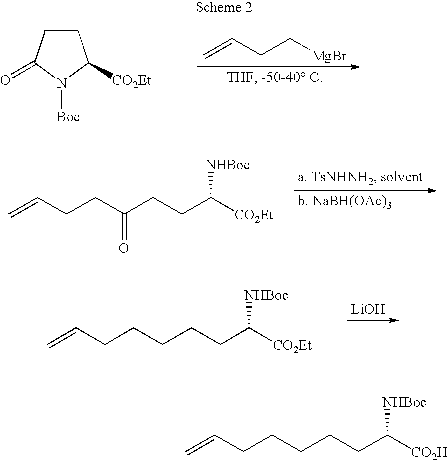

Reduction of the ketone to alcohols of Formula III (A=OH) can be achieved with numerous reagents known to selectively reduce ketones in the presence of esters; sodium borohydride in alcoholic solvents, for example, or lithium (tri-tertbutoxy)aluminum hydride in ethereal solvents. Since the ketone contains a chiral center at C-2, reduction of the alcohol can produce either or both of two possible diastereomers. While the stereochemistry of the reduction depends on both the structure of the starting material and the reduction reaction conditions, one of ordinary skill can readily obtain either diastereomeric alcohol as needed by selecting appropriate reaction conditions, or by chromatographic separation of a mixture of products. Thus the invention contemplates that each diastereomeric alcohol of Formula III (A=OH) is accessible from a ketone of Formula II by standard techniques such as NaBH₄ and DIBALH reductions.

These alcohols can likewise be converted by well known chemistry into derivatives where the alcohol is further functionalized. For example, the alcohols may be acylated by reaction with an anhydride, acyl chloride, or acyl imidazole in the presence of a proton acceptor such as pyridine, catalyzed by dimethylaminopyridine (DMAP) where needed to produce compounds of Formula III where A=—OC(O)R². Similarly, sulfonate analogs (A=—OSO₂R², e.g. mesylates and tosylates) can be prepared by reaction of the alcohols with an appropriate sulfonyl chloride in pyridine, also optionally catalyzed by DMAP. Carbamate derivatives (A=—OC(O)NR³R⁴) can be prepared by reaction of the alcohol with the corresponding carbamyl chloride where available, or, when R⁴ is H, by reaction of the alcohol with an appropriate isocyanate (R³—N═C═O). Other groups such as azide (—N₃) or cyanide (—CN) may be introduced by nucleophilic displacement of tosylate or mesylate derivatives of the alcohols with the appropriate nucleophile. Certain substituents can also be introduced in place of the —OH by a Mitsunobu reaction in the presence of carboxylate or thiol nucleophiles, for example. The hydroxyl can be replaced by halogen directly in some cases, e.g. with PBr₃, or indirectly by formation of a mesylate or tosylate followed by reaction with e.g. sodium iodide in acetone. Trialkylsilyl groups can be attached to the hydroxyl oxygen by methods well known in the art, mostly involving reaction of the alcohol with a trialkylsilyl halide or trialkylsilyl triflate in the presence of a proton acceptor such as a trialkylamine or pyridine. Furthermore, amine groups may be introduced instead of an alcohol by reductive amination of the ketone in the presence of sodium cyanoborohydride and a primary or secondary amine. Thus the invention contemplates the further transformation of the ketones of Formula II into derivates of Formula III where A=—OC(O)R², —OC(O)—OR², —OSiR⁵R⁶R⁷, —OC(O)NR³R⁴, —SC(O)R², —OC(O)SR² and —NR³R⁴, having structural similarity to the alcohol but useful, for example, as intermediates to provide access to other biologically active analogs of the hepatitis C drug BILN-2061. The synthesis step is illustrated in Scheme 3 as follows:

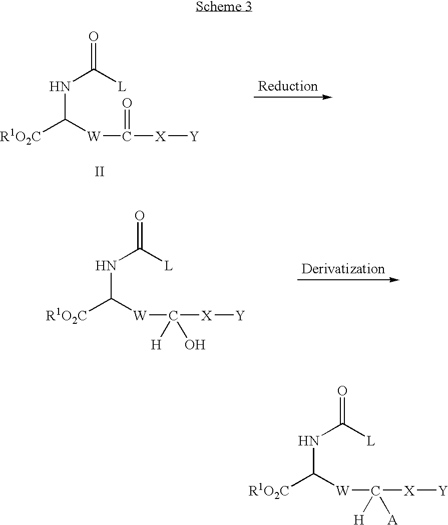

wherein A is —OC(O)R², —OC(O)—OR², —OSiR⁵R⁶R⁷, —OC(O)NR³R⁴, —SC(O)R², —OC(O)SR² or —NR³R⁴.

Likewise, the ketones formed by the ring-opening of N-acyl lactams may be converted into 5- to 7-membered cyclic ketal, thioketal, dithioketal, hemiaminal, or animal derivatives represented by Formula IV:

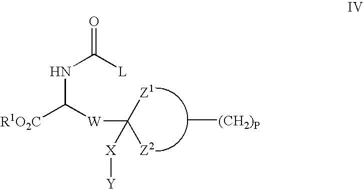

Methods for such conversions are known, and are available, e.g., in the book “Protecting Groups in Organic Synthesis,” 2nd edition, by Greene and Wuts (Wiley and Sons, 1991), which is incorporated herein in its entirety by reference. The general synthesis is illustrated in Scheme 4 as follows:

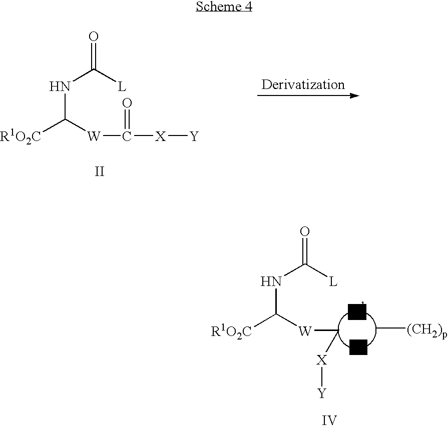

Once the desired compounds of Formula III or Formula IV are prepared, the ester may be hydrolyzed to produce the free carboxylic acid (R¹═H) by standard acidic or basic hydrolysis methods well known in the art. Typically, the hydrolysis would be accomplished with an aqueous solution of a base such as LiOH, NaOH or KOH; alternatively, a dilute solution of HCl or H₂SO₄ may be used. A co-solvent such as THF or an alcohol may be added where the starting ester is not sufficiently soluble in water alone. The hydrolysis reaction is generally conducted at room temperature or an elevated temperature up to the reflux temperature of the solvent employed. Typical reaction times are from about one to about 12 hours.

Similarly, ketones of Formula II can be converted into oxime and alkoxyimine derivatives by methods well known in the art, such as treatment of the ketone with an alkoxyamine, e.g., methoxyamine in a suitable solvent such as ethanol.

### EXAMPLES

Without further elaboration, it is believed that one skilled in the art can, using the preceding description, utilize the present invention to its fullest extent. The following specific examples are intended merely to illustrate the invention and not to limit the scope of the disclosure or the scope of the claims in any way whatsoever.

### Example 1

### Preparation of (2S)—N-Boc-2-amino-non-8-enoic acid

### Step A: Ethyl (2S)—N-Boc-2-amino-5-oxo-non-8-enoate

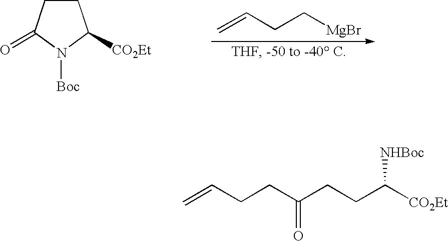

Mg (48 g, 2 mol) and dry THF (1.5 L) were introduced under inert atmosphere into a three-necked flask which was equipped with a dropping funnel and a thermometer. A solution of 4-bromo-1-butene (122 mL, 162 g, 1.13 mol) in dry THF (1.5 L) was introduced into the dropping funnel. About 100 mL of this solution was added first to trigger the reaction. The remaining solution was added dropwise while maintaining the temperature between 60-70° C. (wrapped reaction flask to avoid heat dissipation). When the temperate of the reaction mixture reached room temperature, the reaction was completed. The concentration of the resulting Grignard reagent (3-butenylmagnesium bromide) was 0.37-0.4 M.

To a solution of N-Boc pyroglutamic ethyl ester (269 g, 1.05 mol) in dry THF (6.00 L) which was cooled to between −50 and to −40° C. was added dropwise the Grignard reagent solution (3-butenylmagnesium bromide) (2.80 L, ca. 1.04-1.12 mol). After the addition was completed, the mixture was stirred at this temperature for 30-60 min, then quenched with 10% aqueous NH₄Cl. The organic phase was separated, and the aqueous phase was extracted with ether. The combined organic phase was washed with water, brine, dried (Na₂SO₄), filtered and concentrated under reduced pressure. The residue was chromatographied on silica gel (petroleum/ethyl acetate 5:1) and afforded the desired ketone amino acid ester (194 g, 59%). ¹H NMR (400 MHz, CDCl₃) δ: 5.78 (m, 1H), 5.08 (br, 1H), 5.04-4.96 (m, 2H), 4.22 (br, 1H), 4.18 (q, J=7.2 Hz, 2H), 2.50 (m, 3H), 2.33 (m, 2H), 2.31 (m, 1H), 1.89 (m, 1H), 1.43 (s, 9H), 1.27 (t, J=7.2 Hz, 3H); MS (E/Z): 314 (M+H⁺).

### Step B & C: Ethyl (2S)—N-Boc-2-amino-non-8-enoate

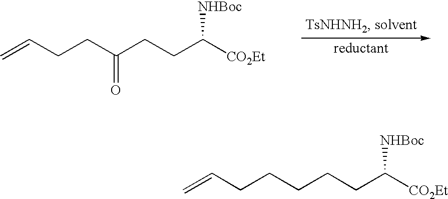

To a solution of ketone (101 g, 0.32 mol) in acetic acid (950 mL) was added p-toluenesulfonyl hydrazide (71.4 g, 0.38 mol), the mixture was stirred at room temperature for 1 h, then NaBH(OAc)₃ (283.8 g, 1.34 mol) was added and stirred 12-24 h (monitored by TLC). After the reaction was completed, the mixture was poured into cold water (6 L) and extracted with ethyl acetate or petroleum ether (4×600 mL). The combined organic phase was washed with saturated aqueous NaHCO₃, brine, dried (Na₂SO₄), filtered, and evaporated under vacuum to afford a crude product, which was chromatographed on silica gel (petroleum ether/ethyl acetate 7:1) to afford ethyl (2S)—N-Boc-2-amino-non-8-enoate (65 g, 67%). ¹H NMR (400 MHz, CDCl₃) δ: 5.78 (m, 1H), 5.00 (br, 1H), 4.96-4.91 (m, 2H), 4.23 (br, 1H), 4.19 (m, 2H), 2.02 (m, 2H), 1.77 (m, 1H), 1.60 (m, 2H), 1.41 (s, 9H), 1.39-1.27 (m, 5H), 1.23 (t, J=7.2 Hz, 3H); MS (E/Z): 300 (M+H⁺).

### Step D: (2S)—N-Boc-2-amino-non-8-enoic acid

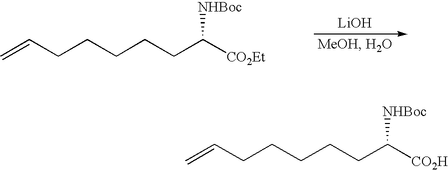

To a solution of ethyl (2S)—N-Boc-2-amino-non-8-enoate (65 g, 0.22 mol) in methanol (500 mL) and H₂O (100 mL) was added LiOH.H2O (44.2 g, 1.08 mol). The mixture was stirred overnight and then concentrated to remove the methanol. The remaining water solution was adjusted to pH=3-5 using 1N HCl and extracted with ethyl acetate (3×200 mL). The combined organic phase was washed with brine, dried (Na₂SO₄), filtered, and evaporated under vacuum to afford (2S)—N-Boc-2-amino-non-8-enoic acid (56 g, 95%). ¹H NMR (400 MHz, CDCl₃) δ: 5.78 (m, 1H), 5.00 (br, 1H), 4.96-4.91 (m, 2H), 4.23 (br, 10H), 2.02 (m, 2H), 1.77 (m, 1H), 1.60 (m, 2H), 1.41 (s, 9H), 1.39-1.27 (m, 5H); MS (E/Z): 272 (M+H⁺); ee value: 99% Daicel Chiralcel AD-H, 0.46*25 cm; n-hexane/isopropanol (7:3), 0.5 mL/min; Rt=12.96 for the S-isomer.

### Example 2

### Preparation of (2R)—N-Boc-2-amino-non-8-enoic acid

### Step A: Ethyl (2R)—N-Boc-2-amino-5-oxo-non-8-enoate

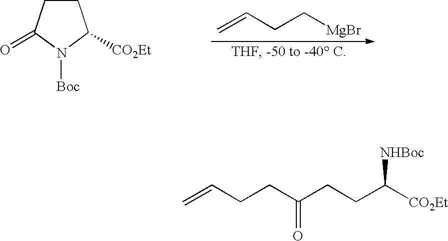

Mg (4.8 g, 0.21 mol) and dry THF (150 mL) were introduced under inert atmosphere into a three-necked flask equipped with a dropping funnel and a thermometer. About 10 ml of the solution of 4-bromo-1-butene (12.2 mL, 16.2 g, 0.113 mol) in dry THF (150 mL) was added to trigger the reaction. Then the remaining solution was added dropwise while maintaining the temperature between 60-70° C. (reaction flask wrapped to avoid heat dissipation). The reaction was completed when the mixture dropped to room temperature. The concentration of the resultant Grignard reagent (3-butenylmagnesium bromide) was around 0.37-0.4 M.

To a solution of N-Boc pyroglutamic ethyl ester (5.14 g, 20 mmol) in dry THF (50 mL) which was cooled to between −50° C. and −40° C. was added dropwise approximately one equivalent amount (50 mL) of the above Grignard reagent (3-butenylmagnesium bromide). After the addition was completed, the mixture was stirred at this temperature for 30-60 min, then quenched with 10% aqueous NH₄Cl. The organic phase was separated, and the aqueous phase was extracted with ether. The combined organic phase were washed with water, brine, dried (Na₂SO₄), filtered and concentrated under reduced pressure. The result residue was chromatographed on silica gel (petroleum/ethyl and acetate 5:1) afforded the desired ketone amino acid ester (4.52 g, 72%). MS (E/Z): 314 (M+H⁺).

### Step B & C: Ethyl (2R)—N-Boc-2-amino-non-8-enoate

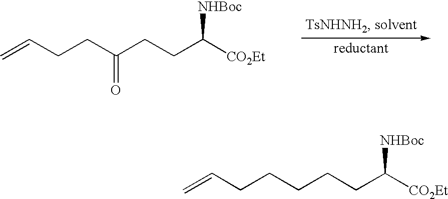

To a solution of ketone (3.14 g, 10 mmol) in acetic acid (50 mL) was added p-toluenesulfonyl hydrazide (2.0 g, 10.8 mmol); the mixture was stirred at room temperature for 1 h, then NaBH(OAc)₃ (12.7 g, 60 mol) was added and stirred for 12-24 h (monitored by TLC). When the intermediate disappeared the reaction mixture was poured into cold water (200 mL) and extracted with ethyl acetate or (petroleum ether) (4×100 mL). The combined organic phase was washed with saturated aqueous NaHCO₃, brine, dried (Na₂SO₄), filtered, and evaporated under vacuum to afford a crude product, which was chromatographied on silica gel (petroleum ether/ethyl acetate 7:1) and afforded ethyl (2R)—N-Boc-2-amino-non-8-enoate (2.06 g, 69%). MS (E/Z): 300 (M+H⁺).

### Step D: (2R)—N-Boc-2-amino-non-8-enoic acid

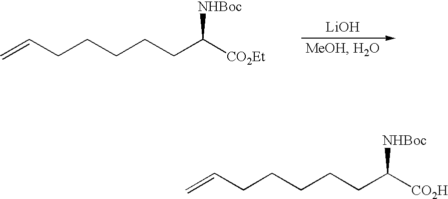

To a solution of the mixture of ethyl (2R)—N-Boc-2-amino-non-8-enoate (2.0 g, 6.7 mmol) in methanol (20 mL) and H₂O (4 mL) was added LiOH.H₂O (1.37 g, 33.5 mmol). The mixture was stirred for overnight and then concentrated to remove methanol. The remained water solution was adjusted to pH=3-5 using 1N HCl and extracted with ethyl acetate (3×20 mL). The combined organic phase was washed with brine, dried (Na₂SO₄), filtered, and evaporated under vacuum to afford (2R)—N-Boc-2-amino-non-8-enoic acid (1.72 g, 95%). MS (E/Z): 272 (M+H⁺); ee value: 99%, Daicel Chiralcel AD-H, 0.46*25 cm; n-hexane/isopropanol (7:3), 0.5 mL/min; Rt=10.52.

What is claimed is:

1. A process for the preparation of a nonracemic amino acid derivative from an optically active N-acyl lactam of Formula I, 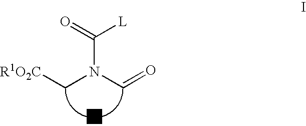
comprising the steps of:
(i) adding an organometallic reagent M-X-Y to a compound of Formula I dissolved in an ethereal solvent to produce a compound of Formula II, 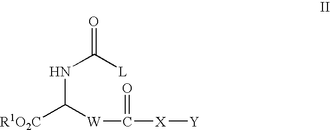
wherein:
M is MgCl, MgBr, MgI, or Li;
R¹ is optionally substituted C₁-C₆ alkyl or optionally substituted C₇-C₁₂ aralkyl;
R⁵ and R⁶ is each independently selected from the group consisting of H, optionally substituted C₁-C₆ alkyl, optionally substituted C₁-C₆ alkoxy, optionally substituted C₇-C₁₂ aralkyl, and optionally substituted phenyl;
R⁷ is optionally substituted C₁-C₆ alkyl, optionally substituted C₁-C₆ alkoxy, optionally substituted C₇-C₁₂ aralkyl, or optionally substituted phenyl;
R⁹ is selected from the group consisting of optionally substituted C₁-C₆ alkyl, optionally substituted C₇-C₁₂ aralkyl, and —SiR⁵R⁶R⁷;
L is selected from the group consisting of tert-butyl, optionally substituted C₁-C₆ alkoxy, and optionally substituted C₇-C₁₂ aralkyloxy;
W is —(CR⁵R⁶)ₙ—, wherein n=2-4;
X-Y is chosen from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4, n=0 to 2;
Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹; and
(ii) reducing the ketone carbonyl of the compound of Formula II to produce a compound of Formula III, 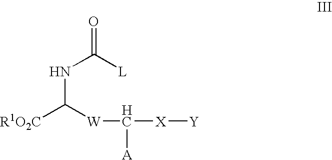
wherein A is H or OH.

2. The process of claim 1 wherein the reduction is effected by using a sulfonyl hydrazine reagent followed by reduction with a reducing reagent to produce a compound of Formula III wherein A is H.

3. The process of claim 2 wherein the sulfonyl hydrazine reagent is a phenyl hydrazine wherein the phenyl ring is optionally substituted, and wherein the reduction is accomplished using a borohydride reducing agent.

4. The process of claim 3 wherein the sulfonyl hydrazine reagent is phenylsulfonyl hydrazine or toluenesulfonyl hydrazine.

5. The process of claim 3 wherein the borohydride reducing agent comprises triacetoxyborohydride.

6. The process of claim 1 where the reduction of the ketone carbonyl produces a compound of Formula III where A is OH.

7. The process of claim 6 where the reduction is accomplished with sodium borohydride.

8. The process of claim 6 where the reduction is accomplished with a chiral borohydride reagent.

9. The process of claim 6 further comprising the step of converting A from OH into a member selected from the group consisting of halogen, —OR², —SR², —NR³R⁴, —N₃, —OSO₂R⁵ and —CN, wherein R² is selected from the group consisting of —C(O)R⁵, —C(O)OR⁵, —C(O)NR³R⁴, and —SiR⁵R⁶R⁷, where R³ and R⁴ are independently selected from the group consisting of H, optionally substituted C₁-C₆ alkyl, optionally substituted C₁-C₆ alkoxy, optionally substituted C₇-C₁₂ aralkyl, and optionally substituted phenyl, or R³ and R⁴ taken together with the N to which they are attached form a five to seven membered ring optionally including one additional heteroatom selected from the group consisting of O and S.

10. The process of claim 6 where X is —(CH₂)ₘ—, Y is unsubstituted vinyl or —C≡C—R⁹, and W is —CHR⁵CHR⁶—.

11. The process of claim 2 where W is —CHR⁵CHR⁶—, wherein R⁵ and R⁶ are independently H or C₁-C₆ alkyl.

12. The process of claim 2 where L is benzyloxy or C₁-C₆ alkoxy.

13. The process of claim 2 where Y is unsubstituted vinyl.

14. The process of claim 12 where X is —(CH₂)ₘ—, Y is unsubstituted vinyl or —C≡C—R⁹, and W is —CHR⁵CHR⁶—.

15. The process of claim 14 where W is —CH₂CH₂— and L is tert-butoxy.

16. The process of claim 15 where m is 2 and Y is unsubstituted vinyl.

17. The process of claim 6 where W is —CH₂CH₂—, X is —(CH₂)ₘ— and Y is unsubstituted vinyl or —C≡C—R⁹.

18. The process of claim 12 where a compound of Formula III is produced in at least 85% e.e.

19. The process of claim 15 where a compound of Formula III is obtained in at least about 85% e.e.

20. A process for the preparation of a nonracemic amino acid derivative of Formula IV: 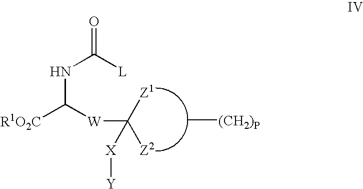
from an optically active N-acyl lactam of Formula I, comprising the steps of:
(i) adding an organometallic reagent M-X-Y to a compound of Formula I 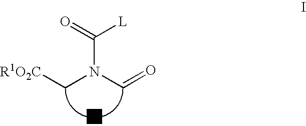
dissolved in an ethereal solvent to produce a compound of Formula II, 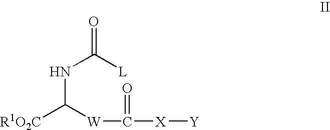
wherein:
M is MgCl, MgBr, MgI, or Li;
R¹ is optionally substituted C₁-C₆ alkyl or optionally substituted C₇-C₁₂ aralkyl;
R⁵ and R⁶ is each independently selected from the group consisting of H, optionally substituted C₁-C₆ alkyl, optionally substituted C₁-C₆ alkoxy, optionally substituted C₇-C₁₂ aralkyl, and optionally substituted phenyl;
R⁷ is optionally substituted C₁-C₆ alkyl, optionally substituted C₁-C₆ alkoxy, optionally substituted C₇-C₁₂ aralkyl, or optionally substituted phenyl;
R⁹ is selected from the group consisting of optionally substituted C₁-C₆ alkyl, optionally substituted C₇-C₁₂ aralkyl, and —SiR⁵R⁶R⁷;
L is selected from the group consisting of tert-butyl, optionally substituted C₁-C₆ alkoxy, and optionally substituted C₇-C₁₂ aralkyloxy;
W is —(CR⁵R⁶)ₙ— where n=2-4;
X-Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4 and n=0 to 2;
Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹; and
(ii) converting the ketone carbonyl of the compound of Formula II to a heterocyclic derivative of Formula IV, wherein Z¹ and Z² are independently selected from the group consisting of O, S, and —NR¹¹, wherein R¹¹ is selected from the group consisting of H, C₁-C₆ alkyl, C₁-C₆ alkoxycarbonyl, and C₂-C₇ acyl, and p=2-4.

21. The process of claim 20 where W is —CHR⁵CHR⁶— and X is —(CH₂)ₘ—.

22. The process of claim 21 where L is C₁-C₆ alkoxy.

23. The process of claim 22 where Y is unsubstituted vinyl or —C≡C—R⁹.

24. The process of claim 23 where W is —CH₂CH₂—.

25. The process of claim 23 where the compound of Formula IV is produced in at least about 85% e.e. at the chiral α-amino acid center.

26. A process for the preparation of an amino acid derivative from an N-acyl lactam of Formula I, 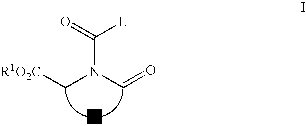
comprising the steps of:
(i) adding an organometallic reagent M-X-Y to a compound of Formula I dissolved in an ethereal solvent to produce a compound of Formula II, 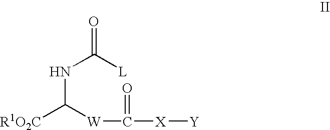
wherein:
M is MgCl, MgBr, MgI, or Li;
R¹ is optionally substituted C₁-C₆ alkyl or optionally substituted C₇-C₁₂ aralkyl;
R⁵ and R⁶ is each independently selected from the group consisting of H, optionally substituted C₁-C₆ alkyl, optionally substituted C₁-C₆ alkoxy, optionally substituted C₇-C₁₂ aralkyl, and optionally substituted phenyl;
R₇ is optionally substituted C₁-C₆ alkyl, optionally substituted C₁-C₆ alkoxy, optionally substituted C₇-C₁₂ aralkyl, or optionally substituted phenyl, and R⁵ and R⁶ are as defined above;
R⁹ is selected from the group consisting of optionally substituted C₁-C₆ alkyl, optionally substituted C₇-C₁₂ aralkyl, and —SiR⁵R⁶R⁷;
L is selected from the group consisting of tert-butyl, optionally substituted C₁-C₆ alkoxy, and optionally substituted C₇-C₁₂ aralkyloxy;
W is —(CR⁵R⁶)ₙ—, wherein n=2-4;
X-Y is selected from the group consisting of —(CH₂)ₘ—Y, —CH₂O—Y, —CH₂S(O)ₙ—Y, —CH₂CH₂CH₂O—Y, —CH₂CH₂CH₂S(O)ₙ—Y, —CH₂—O—(CH₂)ₘ—Y, and —CH₂S(O)ₙ(CH₂)ₘ—Y, wherein m=0 to 4, n=0 to 2; and
Y is selected from the group consisting of optionally substituted vinyl having up to three substituents selected from the group consisting of alkyl, aryl, aralkyl, —OR⁵ and —NR⁵R⁶ with the proviso that not more than one substituent is —OR⁵ or —NR⁵R⁶, optionally substituted aryl, optionally substituted heteroaryl, optionally substituted heterocycle, and —C≡C—R⁹.
(ii) reducing the ketone carbonyl of the compound of Formula II to produce a compound of Formula III, 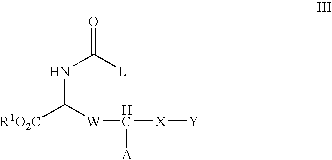
wherein A is H or OH.
